(19) United States
(12) Patent Application Publication
(10) Pub. No.: US 2009/0272386 A1
(43) Pub. Date: Nov. 5, 2009

(54) Nose plug for preventing bruxism

(76) Inventor: David Kurtz, Santa Monica, CA (US)

Correspondence Address: LIU & LIU, 444 S. FLOWER STREET SUITE 1750, LOS ANGELES, CA, 90071, US

(21) Appl. No.: 12/315,734

(22) Filed: Dec. 4, 2008

Related U.S. Application Data
(60) Provisional application No. 61/126,339, filed on May 2, 2008.

(51) Int. Cl. A61F 5/56

(52) U.S. Cl. 128/848

(57) ABSTRACT

A nose plug has two nostril plugs and a bridge section interconnecting the two plugs. The nostril plugs has a generally symmetrical body along an extending axis, having sides that are tapering or bulging, to better conform to nostril cavities. The nostril plugs are shaped, sized and structured from a resilient material (e.g., open or closed cell foam), which can be compressed and inserted into and filling the cavities of the nostrils of a patient to seal the breathing air passages through the nose. The nostril plugs are securely lodged in the nostrils under expansive force of the resilient material. The nose plug device may be constructed with the nostril plugs and bridge section connected in a monolithic structure.

### CROSS REFERENCE

This application claims priority of U.S. Provisional Patent Application No. 61/126,339 filed May 2, 2008, which is fully incorporated by reference as if fully set forth herein.

### BACKGROUND OF THE INVENTION

1. Field of the Invention

The present invention relates to a device for preventing bruxism, and in particular sleep bruxism.

2. Discussion of Related Art

Bruxism is the medical term that refers to an incessant grinding, gnashing and/or clenching of the teeth, unintentionally, involuntarily and/or subconsciously. Psychological factors of stress, tension, anger and anxiety are often the root cause, though other medical and dental conditions can promote it as well. Bruxers (persons with bruxism) are often unaware that they have developed this subconscious habitual conduct. Left untreated, bruxism can cause serious health problems, including damage to the teeth such as weakened teeth, cracked teeth and worn teeth (e.g., dentin exposure), and jaw and facial pains, tense facial and jaw muscles, headaches, earaches, jaw disorders, and other symptoms, discomforts and unpleasant consequences. Nocturnal or sleep bruxism occurs during sleep. This type of bruxism is especially problematic because it often is not detected until after severe damage to the teeth. Nighttime bruxism affects a considerable percentage of both children and adults worldwide.

Treatment options for bruxism may include stress management, dental and other wearable appliance, behavior therapy, and medication. For example, U.S. Pat. No. 5,587, 562 discloses a tooth guard which includes a pressure sensitive surface which is electrically coupled to an alarm mechanism. The device attaches to release clips bonded to the side of a tooth with a sensing area positioned between the teeth so as to indicate bruxism is occurring. The alarm mechanism will provide a resonant frequency capable of making a sound or providing a vibration indicating to the individual that bruxism is occurring. U.S. Pat. No. 6,164,278 discloses a biofeedback modality for the treatment of bruxism, which includes a specially-constructed dental appliance and capsule, which releases a liquid into the mouth when the patient bruxes. U.S. Patent Publication US20080115792A1 discloses a device for preventing bruxism that comprises a carrier intended for receiving in a mouth of a user. The carrier comprises at least a part of an electronic biofeedback system. U.S. Patent Publication US20080006267A1 discloses a device for treating bruxism while sleeping. The nostrils of a nose of a patient are blocked by means of an external clip over the nose, thereby preventing the patient from breathing through the nostrils. As the patient sleeps, the nostrils remain blocked, whereby the patient is forced to breathe through the mouth. Prolong use of the nose clips could risk permanent damage and disfigurement to the nasal cartilage.

The afore-disclosed devices for preventing bruxism involve hardware that is rather uncomfortable when worn by a patient. For example, the nose clips work by pinching the patient's nostrils closed and holding them shut for the full duration of sleep, causing discomfort to the nose at their point of contact. They may further cause greater discomfort should the patient rolls over onto her face, which may also dislodge the nose clips entirely, rendering them ineffective, while presenting a danger to the eyes and face, and a choking hazard. Other devices are costly and largely ineffective. For example, custom fit “bite guards” prescribed by dentists are relatively expensive and prevent physical damages to the teeth. However, they do nothing to relieve the painful and destructive behavior of teeth clenching, and do not stop the pervasive behavior altogether other than just mediate damage.

What is needed is a simple device that is comfortable, safe and effective in preventing bruxism.

### SUMMARY OF THE INVENTION

The present invention provides an improved device that can be worn by a bruxer in reducing or preventing bruxism, without the complication, hazards and discomfort associated with the prior art devices. In one aspect of the present invention, the inventive device is in the form of a nose plug comprising two nostril plugs and a bridge section interconnecting the two plugs. The nostril plugs have a generally symmetrical body along an extending axis. It is preferred that the body of the nostril plugs has sides that are tapering or bulging, to better conform to nostril cavities. The nostril plugs are shaped, sized and structured from a resilient material, which can be compressed and inserted into and fill the cavities of the nostrils of a patient to seal the breathing air passages through the nose. The nostril plugs are securely lodged in the nostrils under expansive force of the resilient material.

With the nostril plugs in place sealing the air passages of the nostrils, airflow is blocked through the nose, thereby forcing the patient to instinctively breathe through her mouth. As the patient breathes through the mouth, the mouth is kept slightly open with the jaws apart, thus preventing touching and/or clenching of the upper and lower sets of teeth, thereby preventing bruxism. The bridge section prevents aspiration of the nostril plugs too deep into the nostrils and respiratory passages, which could cause choking. With extended wearing of the device, it is possible that some patients may be trained and/or conditioned to eventually break the unconscious habit of teeth grinding and clenching altogether.

In one embodiment, the inventive nose plug device is constructed with the nostril plugs and bridge section connected in a monolithic structure. In another embodiment, the device may be constructed with the nostril plugs and bridge section connected by attachments and/or joints (e.g., adhesive, micro-welding, etc.). The nostril plugs and bridge section may be made of a single material, or different materials connected together in a monolithic or joined construction. In one embodiment, the nostril plugs may be made of a foam material (e.g., an open-cell or closed cell foam material). The nose plug device (i.e., the nostril plugs and bridge section) may take various shapes and sizes to accommodate applications to nostrils of various shapes and sizes, with the resiliency of the nostril plugs providing comfortable fitment of a plug of a particular shape and size to nostrils of a range of shapes and sizes. Inserts may be provided in the nostril plugs and/or the base including the bridge section to improve stability of the support for the outer resilient material of the nostril plug.

### BRIEF DESCRIPTION OF THE DRAWINGS

For a fuller understanding of the nature and advantages of the invention, as well as the preferred mode of use, reference should be made to the following detailed description read in conjunction with the accompanying drawings. In the following drawings, like reference numerals designate like or similar parts throughout the drawings.

FIG. 1A is a front view of a nose plug in accordance with one embodiment of the present invention; FIG. 1B shows the nose plug in a compressed state; FIG. 1C is a side view thereof; FIG. 1D is a top view thereof.

FIG. 2 is an illustration of the application of the nose plug to a patient.

FIG. 3A is a front view of a nose plug in accordance with another embodiment of the present invention; FIG. 3B is a top view thereof.

FIG. 4A is a front view of a nose plug in accordance with another embodiment of the present invention; FIG. 4B is a top view thereof.

FIG. 5A is a front view of a nose plug in accordance with another embodiment of the present invention; FIG. 5B is a top view thereof.

FIG. 6A is a front view of a nose plug in accordance with another embodiment of the present invention; FIG. 6B is a top view thereof.

FIG. 7A is a front view of a nose plug in accordance with another embodiment of the present invention; FIG. 7B is a top view thereof.

FIG. 8A is a front view of a nose plug in accordance with another embodiment of the present invention; FIG. 8B is a top view thereof.

FIG. 9A is a front view of a nose plug in accordance with another embodiment of the present invention; FIG. 9B is a top view thereof.

FIG. 10A is a sectional view of a nose plug in accordance with another embodiment of the present invention; FIG. 10B is a sectional view of a nose plug in accordance with a further embodiment of the present invention; FIG. 10C is a sectional view of a nose plug in accordance with yet a further embodiment of the present invention.

### DETAILED DESCRIPTION OF THE PREFERRED EMBODIMENTS

The present description is of the best presently contemplated mode of carrying out the invention. This description is made for the purpose of illustrating the general principles of the invention and should not be taken in a limiting sense. The scope of the invention is best determined by reference to the appended claims. The invention has been described herein in reference to various embodiments and drawings. It will be appreciated by those skilled in the art that variations and improvements may be accomplished in view of these teachings without deviating from the scope and spirit of the invention.

The present invention provides an improved device that can be worn by a bruxer in reducing or preventing bruxism, without the complication, hazards and discomfort associated with the prior art devices. One aspect of the present invention is a nose plug device that comprises two nostril plugs and a bridge section interconnecting the two nostril plugs. The nostril plugs have a generally symmetrical body about an axis extending from the base of the nostril plugs. The body of the nostril plugs may be straight, bulging or tapering, and are generally sized and shaped to fit snugly in the nostrils of a patient under a compression fit.

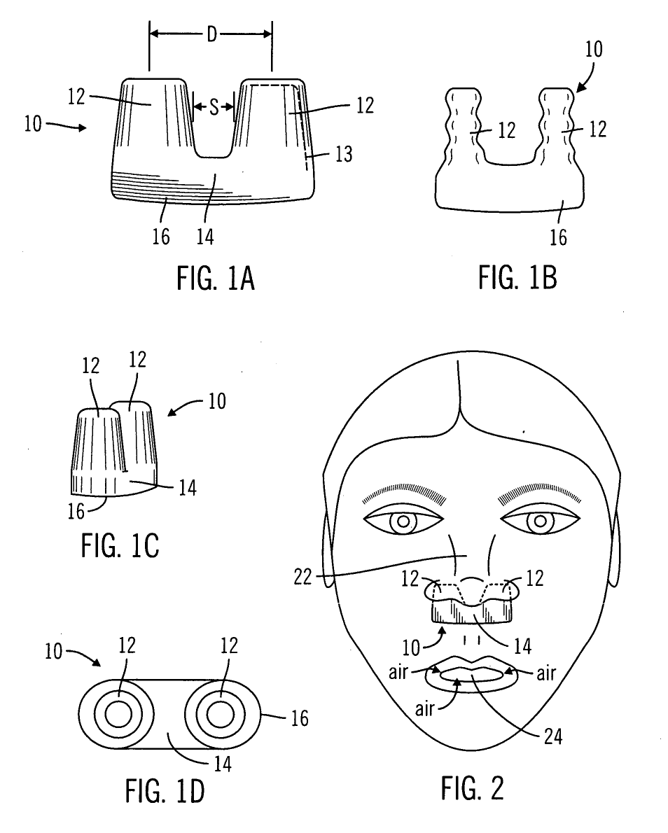

In the embodiment illustrated in FIG. 1, the nose plug 10 includes two nostril plugs 12 spaced apart and connected by a bridge section 14 on base 16. In the embodiment shown in FIG. 1, the body of the nostril plugs 12 is generally conical in shape, extending from the base 16 with gradually narrowly or convergent tapering sides, from wide to narrow. The nostril plugs 12 are spaced apart by the bridge section 14 by an amount similar to the spacing between the nostrils of a typical person. The bridge section 14 has a length that provide a center spacing D between the nostril plugs 12, and a spacing S between the adjacent nostril plugs 12 to accommodate the nostril spacing and nasal columella (the tissue separating two nostrils) of a person. (As will be noted below, the nose plug 10 is made of a resilient material, which allows the nose plug 10 to be applied to a range of nostril sizes and spacings for a given D and S for a particular nose plug 10.) The base 16 may be integral to the nostril plugs 12, such that the nostril plugs 12 are simply interconnected by the bridge section 14.

In the illustrated embodiment, the nose plug is constructed with the nostril plugs 12 and the base 16 including the bridge section 14 in a monolithic structure, for example pressure molded from the same foam material. The nose plug 10 is shaped, sized and structured from a same resilient material. The resilient material may be polyurethane foam, in particular open cell, slow recovery polyurethane foam. The outer surface of the nostril plugs 12 may be structured to resist absorption of fluids. For example, the surface may be structure or include a skin layer 13 of material that resist nasal fluid and soiling, which can be formed as a continuous, substantially closed or non-porous skin created in the pressure-molding process. Remaining tiny pores on the surface are largely impervious to fluid contamination. The foam material may incorporate additives that are anti-bacterial, which is desirable for nose plugs to be reusable.

The material of the nostril plugs 12 and the base 16 including the bridge section 14 may be the same material, or the material of the nostril plugs may be different from the material of the base and bridge section. For example, the material of the nostril plugs can be open cell or closed cell plastic foam, and the base and bridge section can be of a harder material, such as plastic or metal, which preferably is flexible, but not necessarily as resilient as the nostril plugs 12, and which may have an outer layer of resilient material. Even when the nostril plugs and the base and bridge section are made from different materials, they can be connected together in a monolithic structure (e.g., integrally formed, or by molding parts of different plastic materials). In another embodiment (not shown), the nose plug may be constructed with the nostril plugs connected to a separate piece of base including bridge section, by attachments and/or joints (e.g., adhesive, micro plastic-welding, threaded attachment, etc.). Further, the base may be constructed with an insert of a harder material and an outer layer of resilient material, to which base the resilient nostril plugs are attached.

Referring also to FIG. 2, the nostrils plugs 12 are compressed and inserted into and filling the cavities of the nostrils of the nose 22 of a patient 20 to seal the breathing air passages through the nose. FIG. 1A shows the nostril plugs 12 in the fully expanded state before they are inserted into the nostrils of a patient 20. FIG. 1B shows the nostril plugs 12 in a compressed state. After the nostril plugs 12 are inserted into the nostrils in their compressed state, the resilient material slowly expands to recover their shape. The nostril plugs 12 are securely lodged in the nostrils under expansive force of the resilient material, thus forming a compression fit in the nostrils. With the nostril plugs 12 in place sealing the air passages of the nostrils, airflow is blocked through the nose, thereby forcing the patient 20 to instinctively breathe through her mouth 24. As the patient 20 breathes through the mouth 24, the mouth 24 is kept slight open with the jaws apart, thus preventing touching and/or clenching of the upper and lower sets of teeth, thereby preventing bruxism. The bridge section 14 prevents aspiration of the nostril plugs 12 too deep into the nostrils and respiratory passages to cause choking. With extended wearing of the device, it is possible that some patients may be trained and/or conditioned to eventually break the unconscious habit of teeth grinding and clenching altogether.

The soft material of the nose plug 10 provides a comfortable fit that would not damage nasal tissues. The soft material would not cause damage to nasal, eye, and facial tissues should it dislodge from the nostrils. Should the patient roll onto her face during her sleep and her nose is pressed, the soft nostril plugs would yield its shape to the external pressure, without causing discomfort to the patient. The resiliency of the nostril plugs provides a more secure fit in the nostrils to continue to provide the necessary blockage of the nostril passage.

The nose plug 10 may be removed from the nostrils by pulling on the bridge section 14 or the base of the nostrils plugs 12. The slow recovery foam material is soft and pliable enough to yield its shape and position in the nostrils to deliberate force attempting to remove it, but provides a secure seal in the nostrils under normal wear condition, such as during sleep.

The nose plug device 10 (i.e., the nostril plugs 12, base 16 and bridge section 14) may take on various shapes and sizes to accommodate applications to nostrils of various shapes and sizes, with the resiliency of the nostril plugs providing comfortable fitment of a nose plug of a particular shape and size to nostrils of a range of shapes and sizes. The overall dimension of the nostril plugs 12 may be on the order of 0.25″ to 0.75″ wide (or diametric length)×0.5″ to 1.5″ high (axial extension from the base) in the uncompressed state; the dimension of the center distance D between nostril plugs 12 may be on the order of 0.5″ to 1.0″ ; and the dimension of the spacing S between nostril plugs 12 may be on the order of 0.1″ to 0.5″.

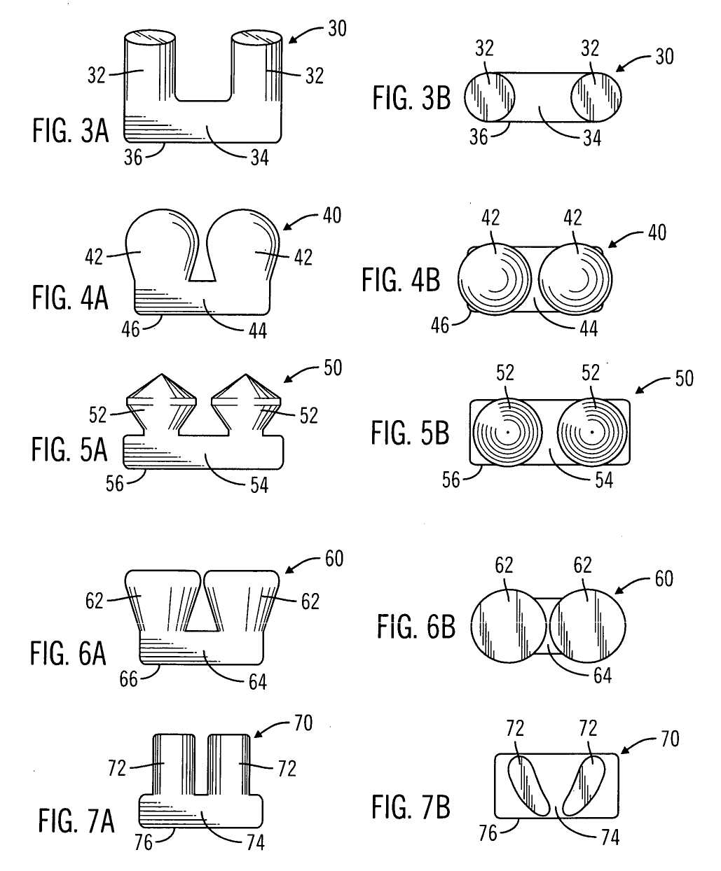

FIGS. 3 to 9 illustrate various additional embodiments of the inventive nose plug.

FIG. 3A is a front view of a nose plug 30 in accordance with another embodiment of the present invention; FIG. 3B is a top view thereof. In this embodiment, the nose plug 30 comprises nose plugs 32 that are generally cylindrical or pillar in shape, which are spaced apart and connected by bridge section 34 on base 36.

FIG. 4A is a front view of a nose plug 40 in accordance with another embodiment of the present invention; FIG. 4B is a top view thereof. In this embodiment, the nose plug 40 comprises nostril plugs 42 that are generally spherical in shape, which are spaced apart and connected by bridge section 44 on base 46.

FIG. 5A is a front view of a nose plug 50 in accordance with another embodiment of the present invention; FIG. 5B is a top view thereof. In this embodiment, the nose plug 50 comprises nostril plugs 52 that have a generally diamond shape, which are spaced apart and connected by bridge section 54 on base 56. The nostril plugs 52 have a body having sides that essentially first taper divergently from the base 56, from narrow to wide, and then taper convergently from the base 56, from wide to narrow.

FIG. 6A is a front view of a nose plug 60 in accordance with another embodiment of the present invention; FIG. 6B is a top view thereof. In this embodiment, the nose plug 60 comprises nostril plugs 62 that are generally conical in shape, which are spaced apart and connected by bridge section 64 on the base 66. Unlike the embodiment in FIG. 1, the narrow ends of the conical nostril plugs are at the base 66 and connected by the bridge section 64 on base 66 (i.e., sides of the body of the nostril plugs 62 taper divergently away from the base 66, from narrow to wide).

FIG. 7A is a front view of a nose plug 70 in accordance with another embodiment of the present invention; FIG. 7B is a top view thereof. In this embodiment, the nose plug 70 comprises nose plugs 72 that are generally ovoid in shape, which are spaced apart and connected by bridge section 74 on base 76. Variation of the ovoid shaped nostril plugs in this embodiment and the spherical shaped nostril plugs in the embodiment of FIG. 4 may include ellipsoidal shaped nostril plugs on base 76.

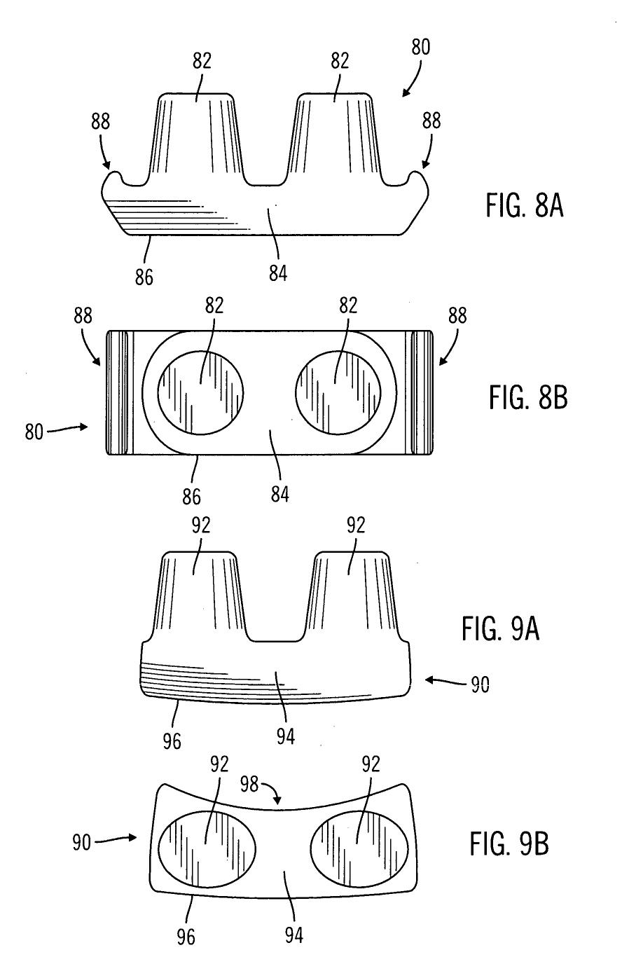

FIG. 8A is a front view of a nose plug 80 in accordance with another embodiment of the present invention; FIG. 8B is a top view thereof. In this embodiment, the nose plug 80 comprises nose plugs 82 that are generally conical in shape as in the embodiment of FIG. 1, which are spaced apart and connected by bridge section 84 on base 86, similar to the embodiment of FIG. 1. However, in this embodiment, a flange 88 is provided at each distal end of the base 86 of the nostril plugs 82. The flanges 88 may be slightly bent or curled upwards away from the plane of the base 86, to provide an improved seal against the side of the nostrils of the patient. Similar flanges may be provided to any of the embodiments of FIGS. 1 to 7, 9 and 10 disclosed herein.

FIG. 9A is a front view of a nose plug 90 in accordance with another embodiment of the present invention; FIG. 9B is a top view thereof. In this embodiment, the nose plug 90 comprises nose plugs 92 that are generally conical in shape as in the embodiment of FIG. 1, which are spaced apart and connected by bridge section 94 on base 96. In this embodiment, the base 96 is concavely curved at one longitudinal edge or side 98 (when viewed from the extended free ends of the nostril plugs 92) to provide a conforming fit to the convex surface of the face of the patient. To provide symmetry, the opposite longitudinal edge or side may be similarly curved. Similar curve(s) in the base may be provided to any of the embodiments of FIGS. 1 to 8 and 10 disclosed herein.

As an alternate embodiment, inserts may be provided in the foam body of the nostril plugs. The insert may be of a stiffer material than the outer foam material, which may be plastic or metal. The inserts may also extend to be part of the base and/or bridge section. While the nostril plugs cannot be compressed as much in this embodiment in the presence of the inserts, additional stability is provided to improve support of the resilient nostril plug material without compromising wear comfort in the nostrils.

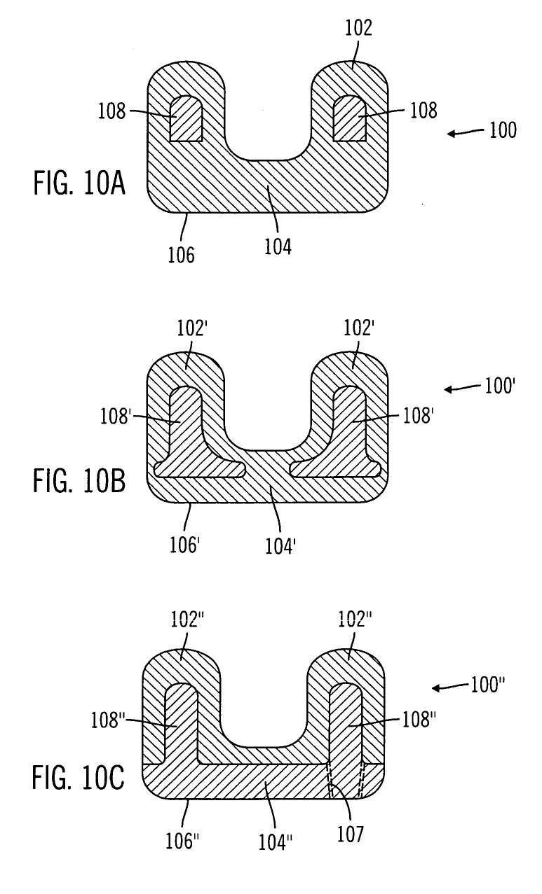

FIG. 10A is a sectional view of a nose plug 100 having inserts in accordance with one embodiment of the present invention, which includes inserts 108 in the nostril plugs 102 that are connected by bridge section 104 on base 106. The inserts 108 may be molded into the nostril plugs 102 as shown in FIG. 10A. The nostril plugs 102 are covered by outer resilient (e.g., foam material) that is similar to the earlier embodiment.

FIG. 10B is a sectional view of a nose plug in accordance with a further embodiment of the present invention, in which the inserts in the nose plug extend to the base 106′. The nostril plugs 102′ and the base 106′ are covered by outer resilient material, but the bridge section 104′ is without insert material. This embodiment allows for flexibility in the bridge section 104′, while providing stability in the base 106′. Alternatively, not shown in the drawings, insert material may be provided within the bridge section 104, but not within the base 106′.

FIG. 10C is a sectional view of a nose plug in accordance with yet a further embodiment of the present invention, in which the inserts 108 within the nostril plugs 102″ extend integrally to the base 106″, but the base is without an outer resilient material, wherein the entire base 106″ including the bridge section 104″ is made of the same material as the insert 108. In this embodiment, the resilient material of the nostril plug 102″ may be provided as a removable cap 103, to be placed over the insert 108″. The removable cap 103 is replaceable with another similar one of similar structure or different structure. This embodiment provides for disposable caps 103 as consumables, which may be made available for purchased separately for use with the same base 106″. This embodiment also allows for making available a base 106″ with several caps 103 of different structures (e.g., sizes and shapes) in a single package, so that the patient can try different caps to find the one that fits best and is most comfortable to the nostrils. It is contemplated within the scope of the present invention to have the insert 108″ made of a material different and/or a separate piece removably attached to the base 108″. For example, the insert 108″ may be joined to the base 106″ by a threaded attachment schematically shown as 107 in FIG. 10C (e.g., the insert 108″ is male threaded and the base 106″ is provided with a hole having complementary female threads), so that inserts 108″ of various sizes and shapes may be provided to allow the patient to try different inserts to match with different size and shape caps 103 to obtain the best overall fit and comfort to the nostrils. Alternatively, the base may have an insert, but the nostril plugs are entirely of a resilient material without any insert.

While the invention has been particularly shown and described with reference to the preferred embodiments, it will be understood by those skilled in the art that various changes in form and detail may be made without departing from the spirit, scope, and teaching of the invention. A person skilled in the art will recognize that the instrument incorporating the essence of this invention can also be used for storage of other types of collapsible items made from plastic or other soft and/or flexible materials. Accordingly, the disclosed invention is to be considered merely as illustrative and limited in scope only as specified in the appended claims.

1. A nose plug device for reducing bruxism, comprising:
two nostril plugs; and
a bridge section interconnecting the nostril plugs, with the nostril plugs spaced apart.

2. The nose plug device as in claim 1, wherein the nostril plugs each has a generally symmetrical body along an extending axis.

3. The nose plug device as in claim 1, wherein the nostril plugs each has a body having sides that are tapering or bulging.

4. The nose plug device as in claim 1, wherein the nostril plugs each has a body that is shaped, sized and structured from a resilient material, which can be compressed and inserted into and filling the cavities of the nostrils of a patient, and which are securely lodged in the nostrils under expansive force of the resilient material.

5. The nose plug device of claim 4, wherein the nostril plugs and bridge section are connected in a monolithic structure.

6. The nose plug device of claim 5, wherein the nostril plugs and the bridge section may be made of same resilient material.

7. The nose plug device of claim 6, wherein the resilient material comprises a foam material.

8. The nose plug device of claim 1, wherein the nostril plugs each includes a base on which each nostril plug extends, whereby the bridge section connects the base of each nostril plug.

9. The nose plug device of claim 1, wherein bridge section is part of a base on which the nostril plugs extend, whereby the nostril plugs are connected via the base by the bridge section.

10. The nose plug device of claim 9, wherein the nostril plugs are integrally connected to the base in a monolithic structure.

11. The nose plug device of claim 9, wherein the base comprises a flange extending from each end beyond the nostril plugs.

12. The nose plug device of claim 11, wherein each flange curves away from plane of the base towards the nostril plugs.

13. The nose plug device of claim 9, wherein the base has a side that is concavely curved when view from extended ends of the nostril plugs.

14. The nose plug device of claim 1, wherein each nostril plug comprises an insert.

15. The nose plug device of claim 14, wherein the insert extends to a base to which the bridge section connects.

16. The nose plug device of claim 15, wherein the insert is threaded to the base.

17. The nose plug device of claim 15, wherein the bridge section comprises the insert.

18. The nose plug device of claim 8, wherein the nostril plugs each has a body having sides that tapers divergently away from the base from narrow to wide.

19. The nose plug device of claim 18, wherein the sides of the body further tapers convergently from wide to narrow from the divergent taper.

20. The nose plug device of claim 8, wherein the nostril plugs each has a body having sides that tapers convergently away from the base from wide to narrow.
